(19) United States
(12) Patent Application Publication
(10) Pub. No.: US 2024/0189588 A1
(43) Pub. Date: Jun. 13, 2024

(54) SYSTEMS AND METHODS FOR IMPLANTABLE ELECTRICAL STIMULATION

(71) Applicants: The Brigham and Women's Hospital, Inc., Boston, MA (US); Northeastern University, Boston, MA (US)

(72) Inventors: David J. Segar, Boston, MA (US); Sumientra Rampersad, Boston, MA (US); Joshua D. Bernstock, Boston, MA (US)

(21) Appl. No.: 18/556,345

(22) PCT Filed: Apr. 19, 2022
(86) PCT No.: PCT/US2022/025395

§ 371 Date: Oct. 19, 2023

Related U.S. Application Data
(60) Provisional application No. 63/176,596, filed on Apr. 19, 2021.

(51) Int. Cl. A61N 1/36; A61B 17/86; A61N 1/05; A61N 1/375; A61N 1/378

(52) U.S. Cl. CPC A61N 1/36002; A61B 17/86; A61N 1/0558; A61N 1/36062; A61N 1/36157; A61N 1/37518; A61N 1/378

(57) ABSTRACT

In accordance with one aspect of the disclosure, a system including a plurality of pedicle screws configured to engage a spine of a patient and each including an electrode, a power source coupled to the pedicle screws and configured to provide an electrical current to each electrode, and a controller is provided. The controller is configured to selectively supply power to the pedicle screws to cause a first electrical stimulation to be output in a first direction from a first pedicle screw to a second pedicle screw, and cause a second, electrical stimulation to be output in a second direction from the second pedicle screw to the first pedicle screw.

### CROSS-REFERENCE TO RELATED APPLICATIONS

This application claims priority to and the benefit of the filing date of U.S. Provisional Patent Application No. 63/176,596, filed Apr. 19, 2021, the entire contents of which is incorporated by reference herein.

### STATEMENT REGARDING FEDERALLY SPONSORED RESEARCH

N/A.

### BACKGROUND OF THE INVENTION

Spinal cancers and spinal tumors can occur in and around the spinal column. Certain methods such as radiotherapy, surgical removal, drug treatments, and chemotherapy can be used to treat spinal cancers and/or spinal tumors. Each of these methods have at least one drawback such as high cost (e.g., certain drug treatments) and/or potential side effects (e.g., chemotherapy). It would therefore be desirable to provide systems and methods that improve the treatment.

### SUMMARY OF THE INVENTION

The present disclosure provides systems and methods that provide improved methods for electrically stimulating spinal and perispinal regions that may include cancers and/or tumors. In one non-limiting example, systems and methods are provided for multidirectional stimulation of spinal and perispinal regions using a plurality of implant devices. In accordance with one aspect of the disclosure, a system including a plurality of pedicle screws configured to engage a spine of a patient and each including an electrode, a power source coupled to the pedicle screws and configured to provide an electrical current to each electrode, and a controller is provided. The controller is configured to selectively supply power to the pedicle screws to cause a first electrical stimulation to be output in a first direction from a first pedicle screw to a second pedicle screw, and cause a second electrical stimulation to be output in a second direction from the second pedicle screw to the first pedicle screw.

In accordance with another aspect of the disclosure, a system including a pedicle screw including an electrode and a threading to secure the pedicle screw in a patient, a power source coupled to the pedicle screw, and a controller configured to control operation of the power source to switch between delivering an electrical current from the pedicle screw to receiving an electrical current at pedicle screw is provided.

In accordance with yet another aspect of the disclosure, a system including a first implant device configured to engage a spine of a patient and comprising a first electrode, a second implant device configured to engage the spine of the patient and comprising a second electrode, a power source coupled to the first implant device and the second implant device and configured to provide an electrical current to the first electrode and the second electrode, and a controller is provided. The controller is configured to selectively supply power to the first implant device and the second implant device to cause a first electrical stimulation to be output in a first direction from the first implant device to the second implant device, and cause a second electrical stimulation to be output in a second direction from the first implant device to the second implant device.

In accordance with yet another aspect of the disclosure, a system including a screw including an electrode and a threading configured to secure the screw with the electrode in a fixed position in a patient, a power source coupled to the screw, and a controller configured to control operation of the power source to selectively deliver an electrical current from the electrode or receive an electrical current with the screw to effectuate a therapeutic plan is provided.

The foregoing and other aspects and advantages of the invention will appear from the following description. In the description, reference is made to the accompanying drawings which form a part hereof, and in which there is shown by way of illustration embodiments of the invention. Any such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic diagram of an example of a stimulation system in accordance with the disclosed subject matter.

FIG. 2 is a further schematic diagram of an example of hardware that can be used to implement a controller and a computing device shown in FIG. 1 in accordance with the disclosed subject matter.

FIG. 3 is a perspective view of an example of an implant device in accordance with some aspects of the disclosed subject matter.

FIG. 4 is another perspective view of an example of an implant device in accordance with some aspects of the disclosed subject matter.

FIG. 5 is yet another perspective view of an exemplary implant device having a portion of a threading and an electrode cut away in accordance with some aspects of the disclosed subject matter.

FIG. 6 is an additional perspective view of an implant device in accordance with some aspects of the disclosed subject matter.

FIG. 7 is a cross-sectional view of the implant device in FIG. 6 in accordance with some aspects of the disclosed subject matter.

FIG. 8 is an elevational view of first implant device and a second implant device in accordance with some of the disclosed subject matter.

FIG. 9 is a set of cross-sectional views of a plurality of implant devices oriented to provide multidirectional stimulation in accordance with some aspects of the disclosed subject matter.

FIG. 10 is a perspective view of a first implant device and a second implant device implanted in a vertebra in accordance with some aspects of the disclosed subject matter.

FIG. 11 is an elevational view of a first implant device implanted in a first vertebra and a second implant device implanted in a second vertebra in accordance with some aspects of the disclosed subject matter.

FIG. 12 is a perspective view of a first implant device, a second implant device, a third implant device, and a fourth implant device arranged to provide multidirectional electrical stimulation in accordance with some aspects of the disclosed subject matter.

FIG. 13 is a schematic illustration of a system including a plurality of implant devices -L implanted in a spinal region in accordance with some aspects of the disclosed subject matter.

FIG. 14 is another schematic illustration of a system including a plurality of implant devices -L implanted in a spinal region and a perispinal region in accordance with some aspects of the disclosed subject matter.

FIG. 15 is a further schematic illustration of a system including a plurality of implant devices implanted in a spinal region in accordance with some aspects of the disclosed subject matter.

FIG. 16 is a flow chart providing some non-limiting example steps of a process for providing electrical stimulation to a patient in accordance with some aspects of the disclosed subject matter.

### DETAILED DESCRIPTION OF THE INVENTION

The present disclosure provides systems and methods to control and deliver stimulation to a subject. In one non-limiting example, the systems and methods may be used to electrically stimulate spinal and perispinal regions in subjects. In one non-limiting clinical application, for example, the subjects may have cancer and/or tumors in the spinal column and/or perispinal regions. In another non-limiting clinical application, the subject may have leptomeningeal disease (LMD).

In one non-limiting example, the systems and methods may be used to provide therapeutic modulation to treat neuroinflammation and/or neuroinfection. In one non-limiting example, the systems and methods may be used to provide therapeutic neuromodulation in spinal cord injury and/or dysfunction (e.g., for neural repair and/or plasticity). In another non-limiting example, the systems and methods may be used to increase and/or supplement fusion rates and the modulation and/or engagement of the immune system during immunotherapy.

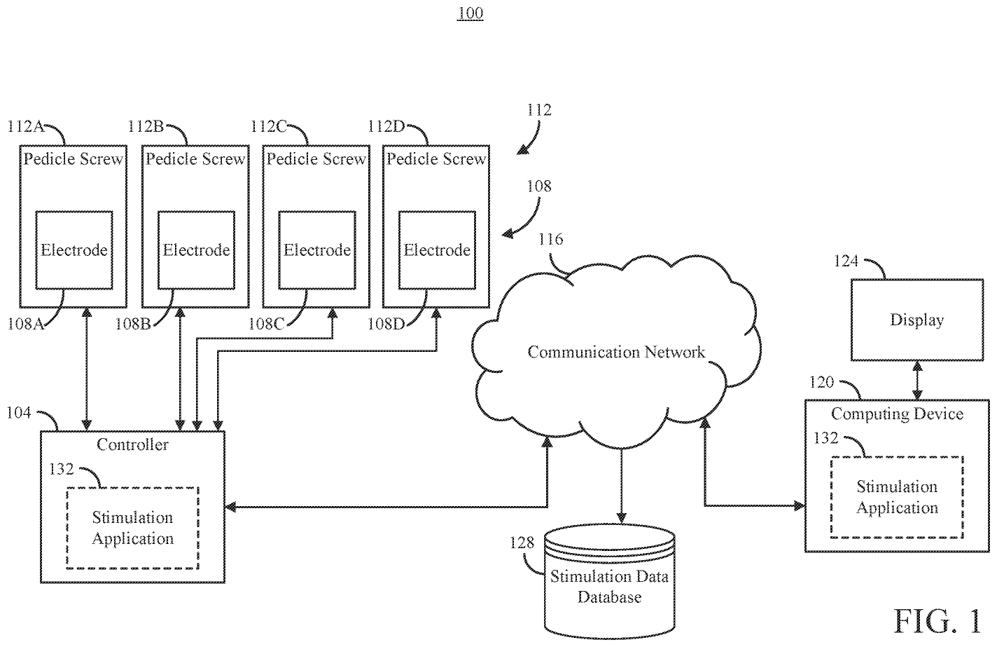

FIG. 1 is a schematic diagram of an example of a stimulation system 100 in accordance with one aspect of the disclosure. In some aspects, the stimulation system 100 can be an implantable stimulation system (e.g., implantable in a patient). Specifically, in some aspects, a controller 104, a plurality of electrodes 108, and a plurality of implant devices 112 can be implanted in a patient. In some configurations, the stimulation system 100 can include the controller 104, the plurality of electrodes 108, the plurality of implant devices 112, a communication network 116, a computing device 120, a display 124, and a stimulation data database 128. In some configurations, the plurality of electrodes can include a first electrode 108A, a second electrode 108B, a third electrode 108C, and a fourth electrode 108D. In some configurations, the stimulation system 100 can include more than four electrodes (e.g. five electrodes, ten electrodes, twenty electrodes, etc.). In some configurations, the controller 104 can be in communication (e.g., wired communication, wireless communication) with the computing device 120, the display 124, and/or the stimulation data database 128. In some configurations, the computing device 120 can be in communication (e.g., wired communication, wireless communication) with the controller 104, the display 124, and/or the stimulation data database 128.

In some configurations, each implant device included in the plurality of implant devices 112 can be a pedicle screw. In some configurations, the plurality of implant devices 112 can include a first pedicle screw 112A, a second pedicle screw 112B, a third pedicle screw 112C, and a fourth pedicle screw 112D. However, it is noted that implant devices 112 are not limited to pedicle screws, and can be implemented in a variety of different manners. For example, implant devices 112 can be cervical lateral mass screws, sacral screws, laminar screws, cortical screws, or other types of screws and/or combinations thereof. Moreover, implant devices 112 can include implants that are not necessarily screws with a head and threads, such as transverse connects, rods, various types of surgical implants, orthopedic implants, staples, and other types of implant devices and combinations thereof. While pedicle screws and various example implant devices are described in detail, it will be appreciated that these other types of implant devices may also be used in a similar manner. Implant devices 112 in some aspects engage the spine of a patient. Implant devices 112 can be implanted in one or more vertebrae in various positions such that implant devices 112 not only contain electrodes 108 to provide a means for therapeutic electrical stimulation of the spine of the patient, but also can function to provide structural support for the spine of the patient.

The electrical stimulation provided by implant devices 112 and electrodes 108 can be in the form of one or more tumor treating fields (TTFs). Tumor treating fields can be provided as therapeutic electrical stimulation using implant devices 112 and electrodes 108 in a variety of ways. For example, tumor treating fields can be alternating electric fields provided at various strengths and frequencies. In some aspects, the tumor treating fields are low-intensity, intermediate-frequency alternating electric fields. The tumor treating fields can serve as mitotic inhibitors that disrupt microtubules and slow cell division. As a result, cells can be destroyed during telophase and cytokinesis.

In some configurations, each electrode included in the plurality of electrodes 108 can be coupled to an implant device included in the plurality of implant devices 112. For example, the first electrode 108A can be coupled to the first pedicle screw 112A, the second electrode 108B can be coupled to the second pedicle screw 112B, the third electrode 108C can be coupled to the third pedicle screw 112C, the fourth electrode 108D can be coupled to the fourth pedicle screw 112D. In some configurations, each electrode included in the plurality of electrodes 108 can form a portion of an outer surface of each pedicle screw included in the plurality of pedicle screws. Further details of exemplary pedicle screws will be discussed further below.

The controller 104 may communicate to the implant device(s) via wired or physical connection or may communicate wirelessly. Additionally or alternatively, the controller 104 can implement portions of a stimulation application 132, which can involve communicating only commands or providing electrical power, current, and/or voltage to one or more of the pedicles screws 112A-D. In some configurations, the stimulation application 132 can involve the controller 104 transmitting and/or receiving instructions, data, commands, etc. from one or more other devices. For example, the controller 104 can receive stimulation commands from the stimulation data database 128 and/or the computing device 120 and/or transmitting stimulation data to the stimulation data database 128 and/or the computing device 120.

As shown in FIG. 1, the communication network 116 can facilitate communication between the controller 104, the computing device 120, and the stimulation data database 128. In some configurations, communication network 116 can be any suitable communication network or combination of communication networks. For example, communication network 116 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some configurations, communication network 116 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, and the like.

In some non-limiting examples, the controller 104 can execute at least a portion of the stimulation application 132 to provide electrical power, current, and/or voltage to the plurality of electrodes 108. The controller 104, the computing device 120, and/or the stimulation data database 128 can include stimulation commands to cause electrical power and/or current to flow between electrodes included in the plurality of electrodes 108. For example, the stimulation commands can include instructions to cause a 100 kHz AC current to flow between the first electrode 108A and the second electrode 108B and/or instructions to cause a 100 kHz AC current to flow between the second electrode 108B and the first electrode 108A. This may be achieved by deliver the 100 kHz AC to the first electrode 108A, or may include a command to draw such current parameters from an energy storage unit or batter that is separate from, integrated with, or coupled to the pedicle screw 112A. In some configurations, the controller 104 can execute the at least a portion of the stimulation application 132 to cause electrical power and/or current to flow between electrodes included in the plurality of electrodes 108 based on the stimulation commands included in the controller 104, the computing device 120 can/or the stimulation data database 128. The stimulation commands can be previously determined based on patient requirements by another system and/or a medical practitioner.

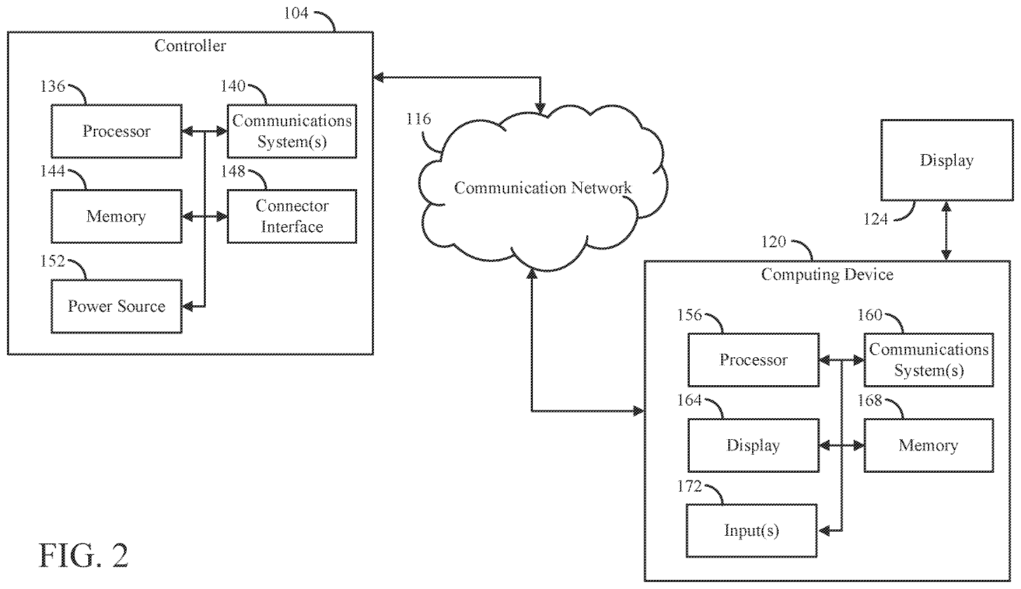

FIG. 2 shows an example of hardware that can be used to implement a controller 104 and a computing device 120 shown in FIG. 1 in accordance with some aspects of the disclosed subject matter. As shown in FIG. 2, the controller 104 can include a processor 136, a communication system 140, a memory 144, a connector interface 148, and a power source 152. The processor 136 can implement at least a portion of the stimulation application 132, which can, for example, be executed from a program (e.g., saved and retrieved from the memory 144). The processor 136 can be any suitable hardware processor or combination of processors, such as a central processing unit (“CPU”), a graphics processing unit (“GPU”), etc., which can execute a program, which can include the processes described below.

In some configurations, the communication system 140 can include any suitable hardware, firmware, and/or software for communicating with the other systems, over any suitable communication networks. For example, the communication system 140 can include one or more transceivers, one or more communication chips and/or chip sets, etc. In a more particular example, the communication system 140 can include hardware, firmware, and/or software that can be used to establish a coaxial connection, a fiber optic connection, an Ethernet connection, a USB connection, a Wi-Fi connection, a Bluetooth connection, a cellular connection, etc. In some configurations, the communication system 140 allows the controller 104 to communicate with the computing device 120 (e.g., directly, or indirectly such as via the communication network 116).

In some configurations, the memory 144 can include any suitable storage device or devices that can be used to store instructions, values, etc., that can be used, for example, by the processor 136 to present content using the display 148 and/or the display 124, to communicate with the computing device 120 via communications system(s) 140, etc. The memory 144 can include any suitable volatile memory, non-volatile memory, storage, or any suitable combination thereof. For example, the memory 144 can include RAM, ROM, EEPROM, one or more flash drives, one or more hard disks, one or more solid state drives, one or more optical drives, etc. In some configurations, the memory 144 can have encoded thereon a computer program for controlling operation of the controller 104 (or the computing device 120). In such configurations, the processor 136 can execute at least a portion of the computer program to present content (e.g., user interfaces, images, graphics, tables, reports, and the like), receive content from the computing device 120, transmit information to the computing device 120, and the like.

In some configurations, the connector interface 148 can include a number of connectors configured to electrically couple the controller 100 to the plurality of electrodes 108. For example, the connector interface 148 can include a plurality of electrical terminals (e.g., positive terminals, negative terminals and/or neutral terminals). In some configurations, the power source 152 can include one or more batteries (e.g., a lithium ion battery).

In some configurations, the processor 136 can be configured to executed instructions stored on the memory 144 to selective provide electrical stimulation between the first electrode 108A, the second electrode 108B, the third electrode 108C, and/or the fourth electrode 108D. More specifically, the processor 136 can cause the power source 152 to selectively supply voltages and/or currents at the connector interface 148 to cause electrical stimulation (e.g., one or more electrical currents) to flow between the first electrode 108A, the second electrode 108B, the third electrode 108C, and/or the fourth electrode 108D. For example, the processor 136 can cause the power source 152 to supply a high (i.e., positive) voltage at the first electrode 108A and a low (i.e., negative or ground) voltage at the second electrode 108B, and cause a current to flow from the first electrode 108A to the second electrode 108B. As another example, the processor 136 can cause the power source 152 to supply a high voltage at the second electrode 108B and a low voltage at the first electrode 108A, and cause a current to flow from the second electrode 108B to the first electrode 108A.

In some configurations, the communications system 140 can communicate (e.g., bidirectionally communicate) with a computing device 136. In some configurations, the computing device 136 can be a laptop computer, a desktop computer, a tablet computer, a smartphone, and/or another device capable of transmitting data to and from the communications system 140. In some configurations, the computational device 120 can transmit data (e.g., stimulation instructions) to the communications system 140. In some configurations, the communications system 140 can transmit data generated by the controller 100 (e.g., information about stimulation performed on a patient) to the communications system 140.

Still referring to FIG. 2, the computing device 120 can include a processor 156, a communication system 160, a display 164, a memory 168, and an input 172. The processor 156 can implement at least a portion of the cardiac image analysis application 140, which can, for example, be executed from a program (e.g., saved and retrieved from the memory 168). The processor 156 can be any suitable hardware processor or combination of processors, such as a central processing unit (“CPU”), a graphics processing unit (“GPU”), and the like, which can execute a program, which can include the processes described below.

In some configurations, the communication system 160 can include any suitable hardware, firmware, and/or software for communicating with the other systems, over any suitable communication networks. For example, the communication system 160 can include one or more transceivers, one or more communication chips and/or chip sets, etc. In a more particular example, the communication system 160 can include hardware, firmware, and/or software that can be used to establish a coaxial connection, a fiber optic connection, an Ethernet connection, a USB connection, a Wi-Fi connection, a Bluetooth connection, a cellular connection, and the like. In some configurations, the communication system 160 allows the computing device 120 to communicate with the controller 104 (e.g., directly, or indirectly such as via the communication network 116).

In some configurations, the display 164 can present a graphical user interface. In some configurations, the display 164 can be implemented using any suitable display devices, such as a computer monitor, a touchscreen, a television, etc. In some configurations, the inputs 172 of the computing device 120 can include indicators, sensors, actuatable buttons, a keyboard, a mouse, a graphical user interface, a touch-screen display, etc. In some configurations, the inputs 172 can allow a user (e.g., a medical practitioner, such as an oncologist, neurologist and/or neurosurgeon) to interact with the computing device 120, and thereby to interact with the controller 104 (e.g., via the communication network 116).

In some configurations, the memory 168 can include any suitable storage device or devices that can be used to store instructions, values, and the like, that can be used, for example, by the processor 156 to present content using the display 164 and/or the display 124, to communicate with the computing device 120 via communications system(s) 160, and the like. The memory 168 can include any suitable volatile memory, non-volatile memory, storage, or any suitable combination thereof. For example, the memory 168 can include RAM, ROM, EEPROM, one or more flash drives, one or more hard disks, one or more solid state drives, one or more optical drives, etc. In some configurations, the memory 168 can have encoded thereon a computer program for controlling operation of the computing device 120 (or the controller 104). In such configurations, the processor 156 can execute at least a portion of the computer program to present content (e.g., user interfaces, images, graphics, tables, reports, and the like), receive content from the controller 104, transmit information to the computing device 120, and the like.

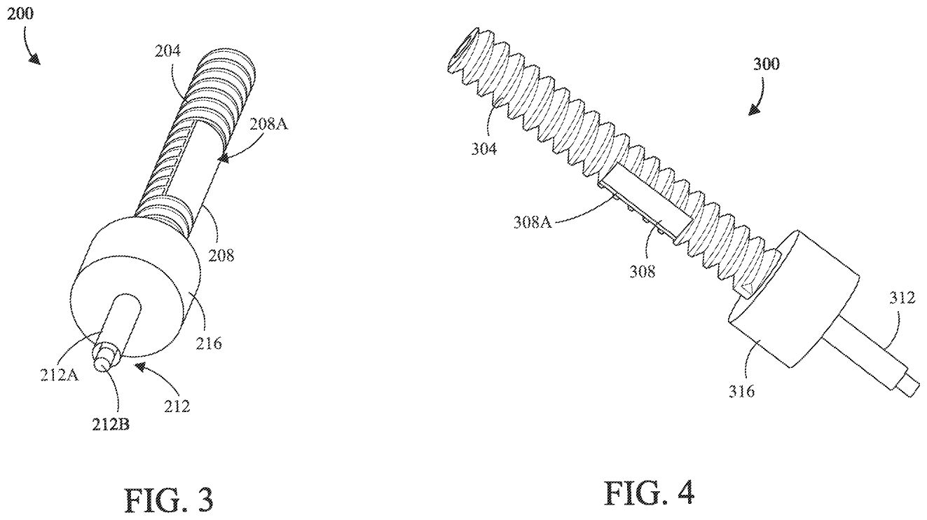

FIG. 3 shows an example of an implant device 200 in accordance with some configurations of the disclosed subject matter. In some configurations, the implant device 200 can be used as one or more of the plurality implant devices 112 in FIG. 1. The implant device 200 can include a threading 204, an electrode 208, and a head 216. The head 216 can be coupled to a cable 212.

The head 216 and the threading 204 can form a body of the implant device. The threading 204 can include threads having a thread pitch and/or thread count suitable for insertion into a portion of a spinal column (e.g., a pedicle) and/or a perispinal region. The head 216 can be gripped during installation of the implant device 200 to allow a user to turn the threading 204 into the spinal column and/or the perispinal region of the patient. In some configurations, the threading 204 and/or the head 216 can be include an insulator material such as a ceramic, a silicone, a plastic, a semiconductor such as titanium dioxide, and/or other materials that can fully insulate the threading 204 and/or the head 216. In some configurations, the insulator material may form a coating on the outer surface of the threading 204 and/or the head 216. In some configurations, the insulator material can form an outer surface as well as a body of the threading 204 and/or the head 216. The insulated material can prevent current from being conducted in areas of the implant device 200 that surround the electrode 208, specifically, the threading 204. In this way, the implant device 200 can provide targeted multidirectional electrical stimulation using the electrode 208, which can provide a greater variety of electrical stimulation as compared to previous methods.

Multidirectional stimulation will be further discussed below. In some configurations, the threading 204 and/or the head 216 can be a conductive material such as stainless steel and/or titanium. The conductive material may allow the electrode 208 to provide targeted electrical stimulation while also allowing the threading 204 to provide supplementary electrical stimulation.

The electrode 208 can form a portion of an outer surface of the implant device 200. In some configurations, the electrode 208 can be a conductive metal such as a stainless steel, tungsten, and/or a titanium alloy. In some configurations, the conductive metal can be used in applications that implant the electrode 208 into bone. In some configurations, the electrode 208 can be an insulated electrode. More specifically, in some configurations, the electrode 208 can be an insulated conductive metal such as a stainless steel, tungsten, and/or a titanium alloy coated with a low impedance insulator such as lead magnesium niobate-lead titanate. The low impedance insulator can allow the electrode 208 to generate a current capacitively without faradaic transfer, which may prevent injury to tissue in contact with the electrode 208. Thus, in some configurations, the electrode 208 can be an insulated conductive metal implanted in soft tissue. The electrode 208 can include an outer surface 208A that is sized to provide selective stimulation in a range of directions. The outer surface 208A can be a substantially smooth surface angularly extending around a portion of the threading 204. In some configurations, the electrode 208 can extend along about ten percent to ninety percent of a length of the threading 204, and angularly extend around about thirty degrees to three hundred and sixty degrees of the outer surface of the threading 204. The electrode 208 can be distanced away from a distal end of the threading 204 to provide appropriate stimulation. In some configurations, the electrode 208 can extend almost to the distal end of the threading 204 (e.g., in applications where the electrode 208 is implanted a cervical spine vertebra). In some configurations, the electrode 208 can extend to about one to three cm away from the distal end of the threading 204 (e.g., in applications where the electrode 208 is implanted a lumbar spine vertebra).

As shown in FIG. 3, electrode 208 can be integrally formed with the threading 204. In some configurations, the electrode 208 can be a portion of the threading 204 in which threads have not been formed (e.g., during manufacturing, threads can be cut into a portion of a cylinder to form the threading 204, and the remaining portion of the threading 204 can form the electrode 208).

In some configurations, the cable 212 can include an outer jacket 212A and a number of conductors 212B. The outer jacket 212A can be an electrical insulator (e.g., an insulative material, such as plastic or rubberized material, for example). The number of conductors 212B can include at least two conductors (e.g., a positive conductor and a neutral conductor) electrically coupled to the electrode 208. The cable 212 can be extend along an inner chamber (not shown) of the implant device 200 from the head 216 to the electrode. The cable 212 can extend outwards from the head 212 to connect to a controller (e.g., the controller 104 in FIG. 1) to receive electrical power, voltage, and/or current.

FIG. 4 shows another example of an implant device 300. The implant device 300 can be one configuration of an implant device 112 as shown in FIG. 1. The implant device 300 can include a threading 304, an electrode 308, and a head 316. The head 316 can be coupled to a cable 312. The head 316 and the cable 312 can be substantially the same as the head 216 and the cable 212 in FIG. 2.

In some configurations, the threading 304 can be nonconductive. For example, the threading 304 can include a conductor coated with an insulator such as coated stainless steel and/or be formed from a nonconductive material. The electrode 308 can be a conductive material (e.g., stainless steel) coupled to an opening in the threading sized to receive the electrode 308. The electrode can include an outer surface 308A that is sized to provide selective stimulation in a range of directions. The outer surface 308A can be a substantially smooth surface angularly extending around a portion of the threading 304.

In some configurations, the electrode 308 can be a substantially wedge-shaped mass of conductive material coupled to the threading 304 (e.g., coupled by one or more welds). The implant device 300 in FIG. 4 may be easier to manufacture than the implant device 200 in FIG. 3. The threading 304 can be formed using a standard threading technique, and an opening sized to house the electrode 308 can be cut into the threading 304. The electrode 308 can then be inserted into the opening and coupled to the threading 304 (e.g., coupled by a welding technique or integrally formed).

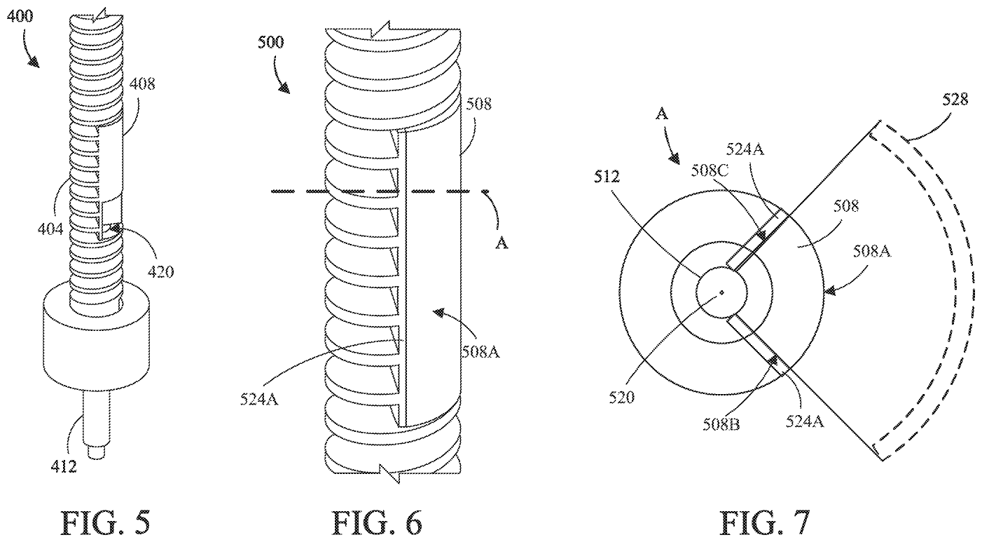

FIG. 5 shows yet another exemplary implant device 400 having a portion of a threading 404 and an electrode 408 cut away. The implant device 400 can be substantially the same as the implant device 200 in FIG. 3. The implant device 400 can include an opening 420 sized to receive one or more conductors included in a cable 412. The opening 420 can have a circular cross-section end extend along a portion of a length of the threading 404.

FIG. 6 shows an additional implant device 500. FIG. 7 shows a cross-section taken along line A of the implant device 500 in FIG. 6. Referring to both FIG. 6 and FIG. 7, in some configurations, the implant device 500 can include a threading 504 and an electrode 508. The threading can include a first sidewall 524A and a second sidewall 524B (shown in FIG. 7) that can prevent movement of the electrode 508 in the threading 504. More specifically, the sidewalls 524A-B can be sized to accept the electrode 508 and impinge on a first side 508A and a second side 508B of the electrode 508 that extend away from an outer surface 508A of the electrode 508. The sidewalls 524A-B can impinge on the first side 508A and the second side 508B of the electrode 508 and provide additional support over a weld, for example.

As mentioned above, the outer surface 508A can angularly extend over a portion of an outer surface of the threading 504. In some configurations, the outer surface 508A can extend over an angle 528 of about eighty degrees. In some configurations, the angle 528 can be selected from about thirty degrees to three hundred and sixty degrees. The electrode 508 can be coupled to a cable 512 included in the implant device 500 in an opening 520 in the threading 504. When coupled to a controller, the electrode can provide electrical stimulation at the outer surface 508A.

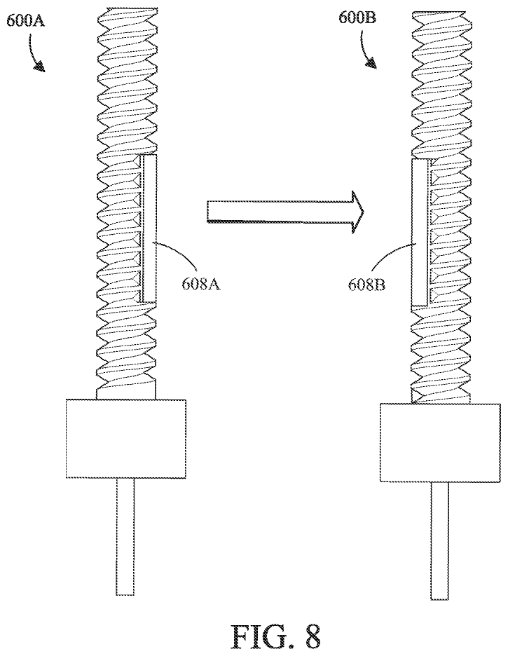

FIG. 8 shows a first implant device 600A and a second implant device 600B. In some configurations, the first implant device 600A and the second implant device 600B can be substantially the same as the implant device 500 in FIG. 5. The first implant device 600A and the second implant device 600B can include a first electrode 608A and a second electrode 608B, respectively. The first electrode 608A can transmit electrical stimulation to the second electrode 608B when the first electrode 608A and the second electrode 608B are generally oriented towards each other. The electrical stimulation can include an AC and/or DC voltage transmitted for a predetermined period of time.

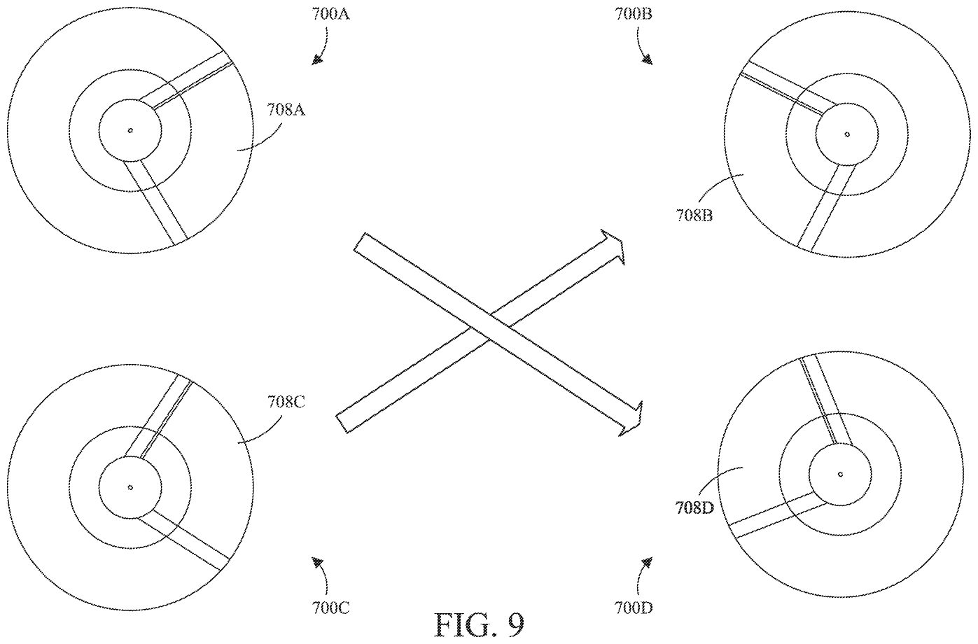

FIG. 9 shows cross-sections of a plurality of implant devices oriented to provide multidirectional stimulation. The plurality of implant device can include a first implant device 700A, a second implant device 700B, a third implant device 700C, and a fourth implant device 700D including a first electrode 708A, a second electrode 708B, a third electrode 708C, and a fourth electrode 708 D, respectively. Each of the first implant device 700A, the second implant device 700B, the third implant device 700C, and the fourth implant device 700D can be substantially the same as the implant device 500 in FIG. 6. In some configurations, the first implant device 700A, the second implant device 700B, the third implant device 700C, and the fourth implant device 700D can be implanted in a spinal region and/or perispinal region of a patient.

The first implant device 700A, the second implant device 700B, the third implant device 700C, and the fourth implant device 700D can be used with a controller (e.g., the controller 104 in FIG. 1) to transmit multidirectional stimulation. In some configurations, electrical stimulation can be transmitted from the first implant device 700A to the fourth implant device 700D, from the third implant device 700C to the second implant device 700B, from the fourth implant device 700D to the first implant device 700A, from the second implant device 700B to the third implant device 700C, from the first implant device 700A to the second implant device 700B, from the second implant device 700B to the first implant device 700A, from the fourth implant device 700D to the third implant device 700C, and/or from the third implant device 700C to the fourth implant device 700D. In some configurations, certain implant devices such as the first implant device 700A and the third implant device 700C may be positioned with the electrodes 708A and 708C sufficiently oriented toward each other in order to transmit electrical stimulation between the first implant device 700A and the third implant device 700C.

In some configurations, each of the electrodes 708A-D can be sufficiently oriented towards at least one other electrode in order to transmit electrical stimulation (e.g., a current) through bodily tissues. In some configurations, depending on a location of a treatment target (e.g., a tumor) and implant locations of the implant devices 700A-D, the orientation of each of the electrodes 708A-D can be chosen to improve transmission efficiency.

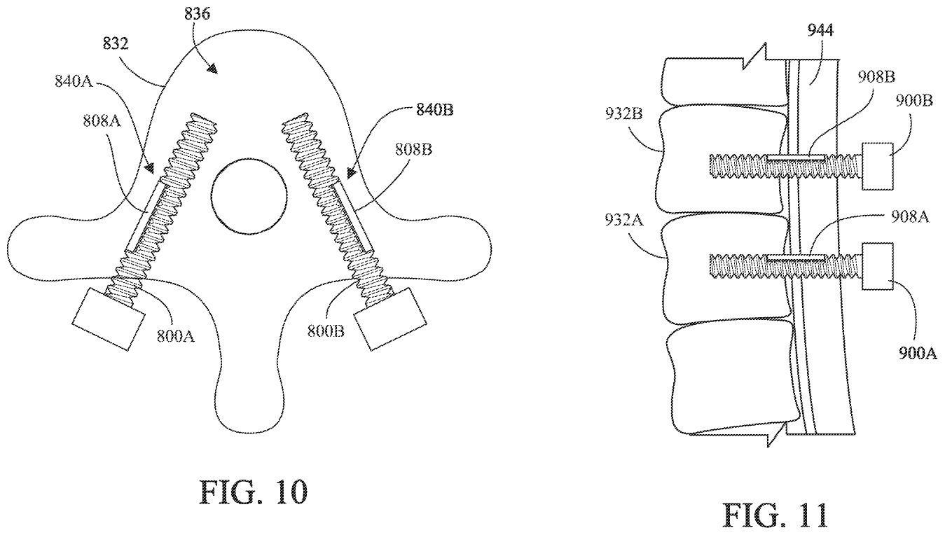

FIG. 10 shows a first implant device 800A and a second implant device 800B implanted in a vertebra 832. The first implant device 800A and the second implant device 800B can include a first electrode 808A and a second electrode 808B, respectively. The vertebra can include a body 836, a first pedicle 840A, and a second pedicle 840B. The first implant device 800A can be coupled to the first pedicle 840A and the body 836. The second implant device 800B can be coupled to the second pedicle 840B and the body 836. During installation of the first implant device 800A and the second implant device 800B, the first electrode 808A and the second electrode 808B can be oriented towards each other in order to allow a controller (not shown) to provide electrical stimulation across the vertebra 832 from the first electrode 808A to the second electrode 808B and/or from the second electrode 808B to the first electrode 808A.

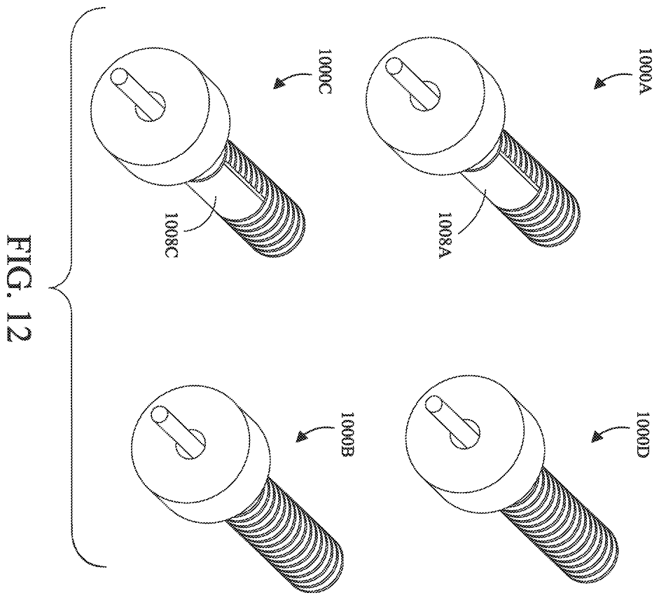

FIG. 11 shows a shows a first implant device 900A implanted in a first vertebra 932A and a second implant device 900B implanted in a second vertebra 932B. The first vertebra 932A and the second vertebra 932B can be arranged around a spinal cord 944 of a patient that may have a cancer and/or a tumor in the spinal cord 944. In some configurations, the patient may have a spinal tumor, an intramedullary tumor, an extramedullary tumor, an intradural tumor, an extradural tumor, a perispinal tumor, and/or a nerve root tumor.

The first implant device 900A can include a first electrode 908A, and the second implant device 900B can include a second electrode 908B arranged to provide electrical stimulation to the spinal cord 944, the first vertebra 932A, and/or the second vertebra 932B.

FIG. 12 shows a first implant device 1000A, a second implant device 1000B, a third implant device 1000C, and a fourth implant device 1000D arranged to provide multidirectional electrical stimulation. The first implant device 1000A can include a first electrode 1008A, the second implant device 1000B can include a second electrode (not shown), the third implant device 1000C can include a third electrode 1008C, and the fourth implant device 1000C can include a fourth electrode (not shown). The first electrode 1008A can be arranged towards the fourth electrode to provide electrical stimulation between the first electrode 1008A and the fourth electrode. The second electrode can be arranged towards the third electrode 1008C to provide electrical stimulation between the second electrode and the third electrode 1008C. In some configurations, the first implant device 1000A and the fourth implant device 1008D can be implanted in a first vertebra to provide multidirectional stimulation in the first vertebra, and the second implant device 1000B and the third implant device 1008C can be implanted in a second vertebra to provide multidirectional stimulation in the second vertebra.

While many of the example implant device described with respect to FIGS. 3-12 are illustrated as screws, it is important to reiterate that a variety of different types of implant devices can be used in accordance with aspects of the disclosure, including implant devices that are not necessarily screws with a head and threads. Implant devices 112, generally, can be cervical lateral mass screws, sacral screws, laminar screws, cortical screws, transverse connects, rods, various types of surgical implants, orthopedic implants, staples, or other types of implant devices and/or combinations thereof.

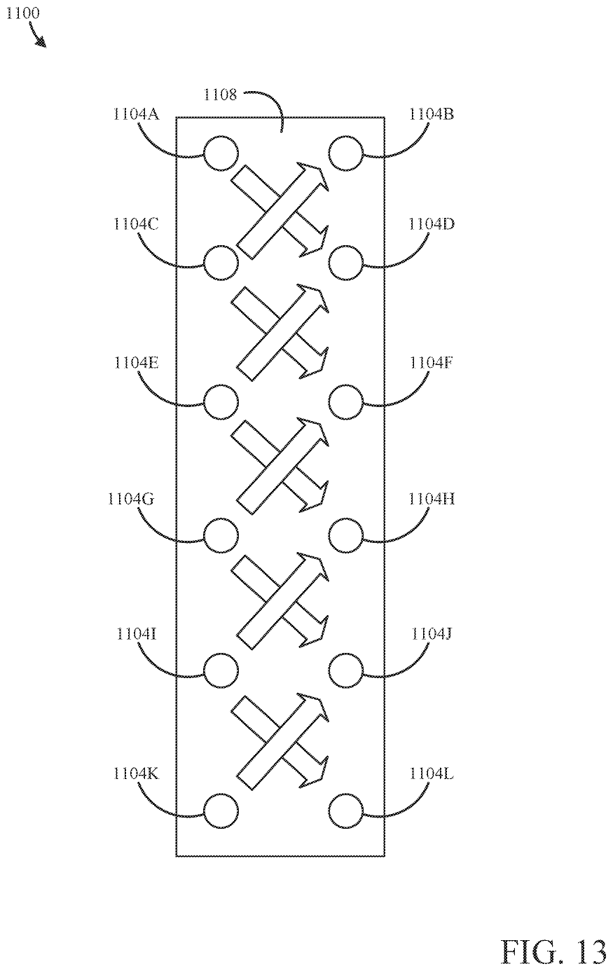

FIG. 13 shows a system 1100 including a plurality of implant devices 1104A-L implanted in a spinal region 1108. Each of the plurality of implant devices 1104A-L can be the implant device 500 in FIG. 6. Each of the implant device included in the plurality of implant devices 1104A-L can be implanted in a vertebra included in the spinal region 1108. In some configurations, the plurality of implant devices 1104A-L can provide electrical stimulation between a first vertebra and a second vertebra included in the spinal region 1108. For example, a first implant device 1104A and a second implant device 1104B can be implanted in a first vertebra, and a third implant device 1104C and a fourth implant device can be implanted in a second vertebra included in the spinal region 1108. The first implant device 1104A and the third implant device 1104C can be oriented towards each other to provide electrical stimulation to and from the first implant device 1104A and the third implant device 1104C. Additionally, the second implant device 1104B and the fourth implant device 1104D can be oriented towards each other to provide electrical stimulation to and from the second implant device 1104B and the fourth implant device 1104D.

Furthermore, the third implant device 1104C can be oriented towards a sixth implant device 1104F coupled to a third vertebra included in the spinal region 1108. The fourth implant device 1104D can be oriented towards a fifth implant device 1104E coupled to the third vertebra. The third implant device 1104B and the sixth implant device 1104F can be oriented towards each other to provide electrical stimulation to and from the third implant device 1104B and the sixth implant device 1104F. The fourth implant device 1104D and the fifth implant device 1104E can be oriented towards each other to provide electrical stimulation to and from the fourth implant device 1104D and the fifth implant device 1104E. Thus, with six implant devices, the system 1100 can provide multidirectional stimulation between three or more levels of vertebra.

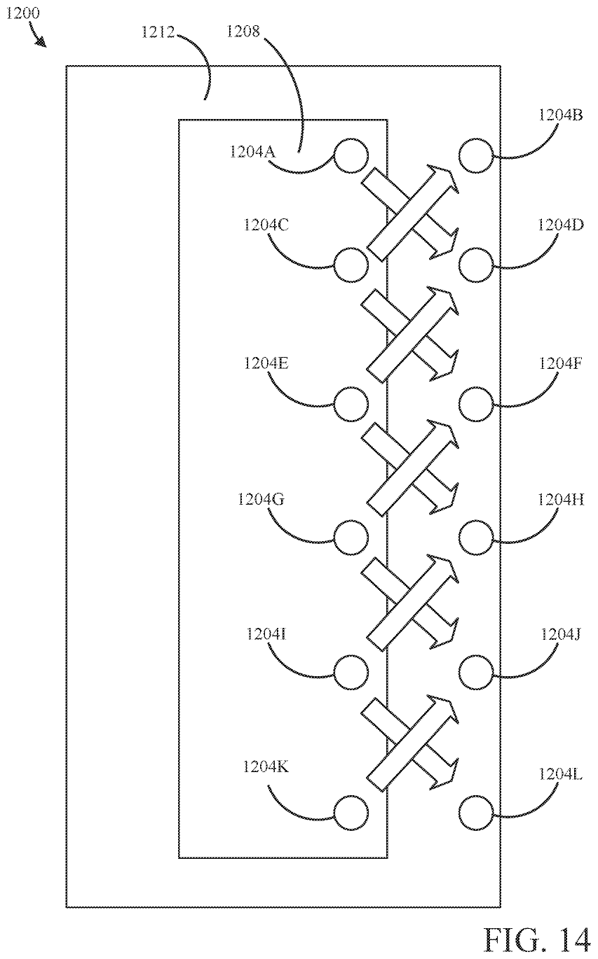

FIG. 14 shows a system 1200 including a plurality of implant devices 1204A-L implanted in a spinal region 1208 and a perispinal region 1212. Each of the plurality of implant devices 1204A-L can be the implant device 500 in FIG. 6. A portion of the implant devices included in the plurality of implant devices 1204A-L can be implanted in a vertebra included in the spinal region 1208, and another portion of the implant devices included in the plurality of implant devices 1204A-L can be implanted in a perispinal region 1212 outside of the spinal region 1208. In this way, the system 1200 can provide electrical stimulation to and from the spinal region 1208 and the perispinal region 1212.

In some configurations, a first implant device 1204A and can be implanted in a first vertebra included in the spinal region 1208, a second implant device 1208B and a fourth implant device 1208D can be implanted in the perispinal region 1212, and a third implant device 1208C can be implanted in a second vertebra included in the spinal region 1208. The first implant device 1204A and the third implant device 1204C can be oriented towards each other to provide electrical stimulation to and from the first implant device 1204A and the third implant device 1204C in both the spinal region 1208 and the perispinal region 1212. Additionally, the second implant device 1204B and the fourth implant device 1204D can be oriented towards each other to provide electrical stimulation to and from the second implant device 1204B and the fourth implant device 1204D in both the spinal region 1208 and the perispinal region 1212.

Furthermore, the third implant device 1204C can be oriented towards a sixth implant device 1204F in the perispinal region 1212. The fourth implant device 1204D can be oriented towards a fifth implant device 1204E coupled to a third vertebra included in the spinal region 1208. The third implant device 1204B and the sixth implant device 1204F can be oriented towards each other to provide electrical stimulation to and from the third implant device 1204B and the sixth implant device 1204F. The fourth implant device 1204D and the fifth implant device 1204E can be oriented towards each other to provide electrical stimulation to and from the fourth implant device 1204D and the fifth implant device 1204E. Thus, with six implant devices, the system 1200 can provide multidirectional stimulation between three or more levels of vertebra and the surrounding perispinal region 1212.

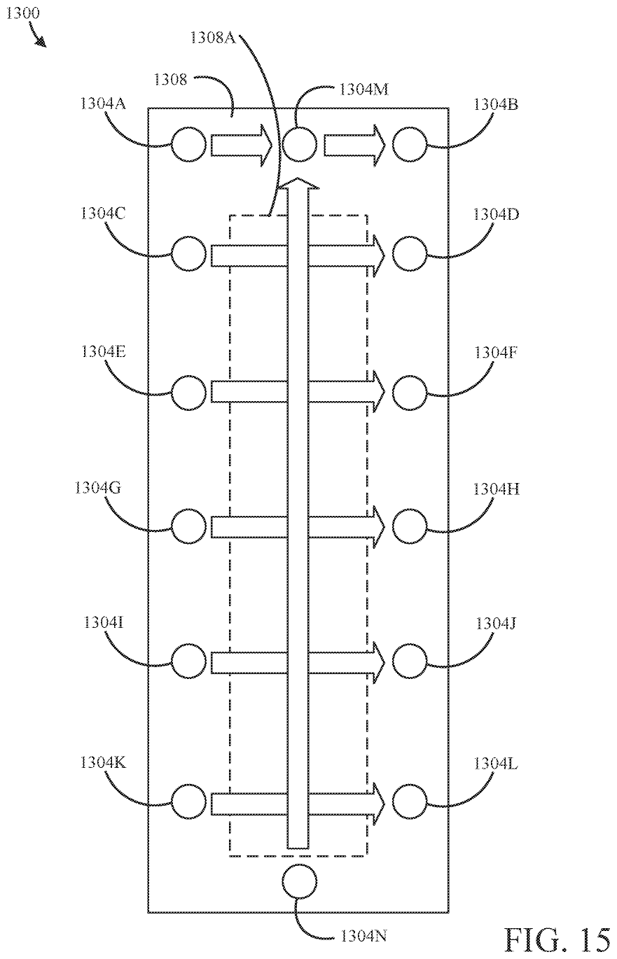

FIG. 15 shows a system 1300 including a plurality of implant devices 1304A-N implanted in a spinal region 1308. Each of the plurality of implant devices 1304A-N can be the implant device 500 in FIG. 6.

The system 1300 can be substantially similar to the system 1100 in FIG. 13 with the addition of a first supplementary implant device 1304M and a second supplementary implant device 1304N, which can be placed at a top and bottom of a spine and/or a spinal cord 1308A, respectively. The supplementary implant devices 1304M and 1304N can provide electrical stimulation along the length of the spine and/or the spinal cord 1308A. Additionally, the first supplementary implant device 1304M can be positioned between the first implant device 1304A and the second implant device 1304B. The first supplementary implant device 1304M may have an electrode sufficiently sized to transmit and/or receive electrical stimulation to and from the first implant device 1304A, the second implant device 1304B, and the second supplementary implant device 1304N. The system 1300 can provide stimulation across the spinal cord 1308A. For example, the third implant device 1304C and the fourth implant device 1304D can be arranged to provide electrical stimulation to and from the third implant device 1304C and the fourth implant device 1304D.

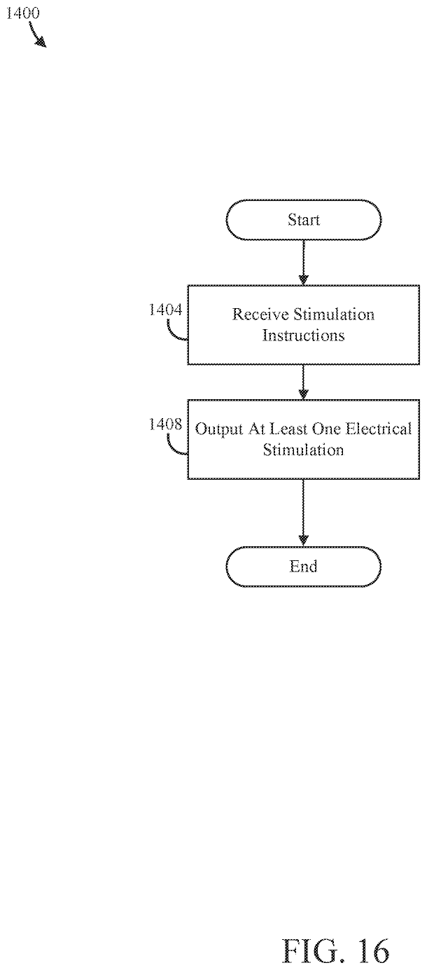

FIG. 16 shows an exemplary process 1400 for providing electrical stimulation to a patient. In some configurations, the process 1400 can be included in the stimulation application 132 in FIG. 1. The process 1400 can be used to treat a patient with a spinal or perispinal tumor such as a spinal tumor, an intramedullary tumor, an extramedullary tumor, an intradural tumor, an extradural tumor, a perispinal tumor, and/or a nerve root tumor.

At 1404, the process 1400 can receive stimulation instructions. The stimulation instructions can include information about electrical stimulation to be transmitted between at least two electrodes included in a plurality of electrodes. The plurality of electrodes can be included in a plurality of implant devices included in a stimulation system (e.g., one of the systems 100 and/or 1100-1300 described above). The stimulation instructions can include at least one voltage and/or current to be provided at one or more electrodes (e.g., by the controller 100) at one or more time points.

At 1408, the process 1400 can output at least one electrical stimulation. The process 1400 can cause the controller output one or more voltages, currents, and power amounts at one or more electrodes based on a portion of the stimulation instructions associated with a current time point. In some configurations, the process 1400 can cause a first electrical stimulation to be output in a first direction from a first pedicle screw to a second pedicle screw and/or cause a second electrical stimulation to be output in a second direction from the second pedicle screw to the first pedicle screw at a first time point. In some configurations, the process 1400 can cause a third electrical stimulation to be output in a third direction from the first pedicle screw to the third pedicle screw to stimulate a perispinal region and/or cause a fourth electrical stimulation to be output in a fourth direction from the second pedicle screw to the fourth pedicle screw to stimulate the perispinal region at a second time point. In some configurations, the process 1400 can cause a third electrical stimulation to be output in a third direction from the third pedicle screw to the fourth pedicle screw to stimulate a patient spinal cord and/or cause a fourth electrical stimulation to be output in a fourth direction from the fourth pedicle screw to the third pedicle screw to stimulate the patient spinal cord at the second time point. The process 1400 can continue to provide electrical stimulation in multiple directions between multiple electrodes for any number of time points until the stimulation instructions are completely executed. The process 1400 can then end.

It is understood that at least some of the steps included in the process 1400 can be rearranged and/or executed in parallel. The process 1400 may be implemented as computer readable instructions on a memory or other storage medium and executed by a processor.

Thus, the present disclosure provides systems and methods for providing multidirectional electrical stimulation to a subject. The systems and methods may be used in spinal and/or perispinal regions in a patient.

The present invention has been described in terms of one or more preferred embodiments, and it should be appreciated that many equivalents, alternatives, variations, and modifications, aside from those expressly stated, are possible and within the scope of the invention.

1. A system comprising:
a plurality of pedicle screws configured to engage a spine of a patient and each comprising an electrode;
a power source coupled to the pedicle screws and configured to provide an electrical current to each electrode; and
a controller configured to selectively supply power to the pedicle screws to:
cause a first electrical stimulation to be output in a first direction from a first pedicle screw to a second pedicle screw; and
cause a second electrical stimulation to be output in a second direction from the second pedicle screw to the first pedicle screw.

2. The system of claim 1, wherein the first pedicle screw is configured to be coupled to a first vertebra, the second pedicle screw is configured to be coupled to a second vertebra, and the first electrical stimulation is directed across the first vertebra.

3. The system of claim 2, wherein the plurality of pedicle screws comprises a third pedicle screw configured to be coupled to a perispinal region of the patient and a fourth pedicle screw configured to be coupled to the perispinal region.

4. The system of claim 3, wherein the controller is further configured to selectively supply power to the plurality of pedicle screws to:
cause a third electrical stimulation to be output in a third direction from the first pedicle screw to the third pedicle screw to stimulate the perispinal region; or
cause a fourth electrical stimulation to be output in a fourth direction from the second pedicle screw to the fourth pedicle screw to stimulate the perispinal region.

5. The system of claim 1, wherein the first pedicle screw is configured to be coupled to a first vertebra, the second pedicle screw is configured to be coupled to a different location within the first vertebra.

6. The system of claim 1, wherein the plurality of pedicle screws comprises a third pedicle screw and a fourth pedicle screw, the first pedicle screw is coupled to a first pedicle in a first vertebra of the spine of the patient, the third pedicle screw is coupled to a second pedicle in the first vertebra, the second pedicle screw is coupled to a first pedicle in a second vertebra of the spine of the patient, and the fourth pedicle screw is coupled to a second pedicle in the second vertebra.

7. The system of claim 6, wherein the first electrical stimulation and the second electrical stimulation are delivered across the first vertebra, and the controller is further configured to selectively supply power to the third pedicle screw and the fourth pedicle screw to:
cause a third electrical stimulation to be output in a third direction from the third pedicle screw to the fourth pedicle screw to stimulate a patient spinal cord; or
cause a fourth electrical stimulation to be output in a fourth direction from the fourth pedicle screw to the third pedicle screw to stimulate the patient spinal cord.

8. The system of claim 1, wherein the first pedicle screw comprises a first electrode having a substantially smooth surface angularly extending across no more than ninety degrees of an outer surface of the first pedicle screw or the second pedicle screw comprises a second electrode having a substantially flat surface angularly extending across no more than ninety degrees of an outer surface of the second pedicle screw.

9. A system comprising:
a first implant device configured to engage a spine of a patient and comprising a first electrode;
a second implant device configured to engage the spine of the patient and comprising a second electrode;
a power source coupled to the first implant device and the second implant device and configured to provide an electrical current to the first electrode and the second electrode; and
a controller configured to selectively supply power to the first implant device and the second implant device to:
cause a first electrical stimulation to be output in a first direction from the first implant device to the second implant device; and
cause a second electrical stimulation to be output in a second direction from the first implant device to the second implant device.

10. The system of claim 9, wherein the first electrode comprises a substantially smooth surface angularly extending across ninety degrees or less of an outer surface of the first implant device.

11. The system of claim 10, wherein the first electrode comprises an electrically conductive metal arranged along a length of the first implant device, and wherein the first electrode is distanced away from a distal end of the first implant device.

12. The system of claim 9, wherein the first electrical stimulation and the second electrical stimulation are configured to treat at least one of a spinal tumor, an intramedullary tumor, an extramedullary tumor, an intradural tumor, an extradural tumor, a perispinal tumor, and/or a nerve root tumor.

13. The system of claim 9, wherein the first implant device is further configured to be secured to a perispinal region of the patient.

14. The system of claim 9, wherein an outer surface of the first implant device comprises an outer surface of the first electrode and a nonconductive material.

15. A system comprising:
a screw comprising an electrode and a threading configured to secure the screw with the electrode in a fixed position in a patient;
a power source coupled to the screw; and
a controller configured to control operation of the power source to selectively deliver an electrical current from the electrode or receive an electrical current with the screw to effectuate a therapeutic plan.

16. The system of claim 15, wherein the power source and the controller operate in concert to carry out the therapeutic plan by deliver the electrical current with parameters selected to treat a tumor in the patient.

17. The system of claim 16, wherein the tumor is at least partially located in a spinal cord of the patient.

18. The system of claim 16, wherein the tumor is at least partially located in a perispinal region of the patient.

19. The system of claim 15, wherein the therapeutic plan comprises providing the electrical current along a length of a spinal cord of the patient.

20. The system of claim 15, wherein the therapeutic plan comprises providing the electrical current across a length of a spinal cord of the patient.

21. The system of claim 15, wherein the screw is configured to at least one of provide or receive electrical currents with at least two other screws secured in the patient.
